(19) United States
(12) Patent Application Publication
(10) Pub. No.: US 2024/0075169 A1
(43) Pub. Date: Mar. 7, 2024

(54) MODEL OF SPINAL CORD HYPOPERFUSION AND ISCHEMIC STROKE WITH IMMEDIATE PARALYSIS

(71) Applicant: Ohio State Innovation Foundation, Columbus, OH (US)

(72) Inventor: Hesham Kelani, Columbus, OH (US)

(21) Appl. No.: 18/459,871

(22) Filed: Sep. 1, 2023

Related U.S. Application Data
(60) Provisional application No. 63/403,044, filed on Sep. 1, 2022.

(51) Int. Cl. A61K 49/00

(52) U.S. Cl. CPC A61K 49/0008

(57) ABSTRACT

The present disclosure relates to methods of inducing central nervous system (CNS) injuries in an animal model for use of identifying and/or screening CNS therapeutic compositions.

### RELATED APPLICATION

This U.S. Utility application claims priority to, and the benefit of, U.S. Provisional Patent Application No. 63/403,044, filed Sep. 1^{st}, 2022, entitled “MODEL OF SPINAL CORD HYPOPERFUSION AND ISCHEMIC STROKE WITH IMMEDIATE PARALYSIS,” which is incorporated by reference herein in its entirety.

### FIELD

The present disclosure relates to methods of inducing central nervous system (CNS) injuries in an animal model for use of identifying and/or screening CNS therapeutic compositions.

### BACKGROUND

Left untreated, thoracic aortic aneurysm disease (incidence, 0.0053%; prevalence, 0.16%) can be lethal. However, both repair methods—traditional open repair and thoracic endovascular aortic repair—cause the severe complication of ischemic spinal cord injury. Although the thoracic stent endograft deployed during thoracic endovascular aortic repair, a less-invasive alternative for abdominal aortic aneurysm since 1991 and thoracic aortic aneurysm since 1994, gained Food and Drug Administration approval in 2005, a clinically relevant mouse model for mechanistic study of thoracic endovascular aortic repair-induced ischemic spinal cord injury has been lacking for over 30 years.

Simulation of thoracic endovascular aortic repair-induced ischemic spinal cord injury has been confined to rabbits, dogs, pigs, and sheep, which presented the same utility issues found in the large-animal open repair models, making them ill-suited for the elucidation of the neuropathological mechanisms of thoracic endovascular aortic repair-induced post-ischemia hypoperfusion.

Given the pitfalls of traditional thoracic aortic aneurysm repair models, there is need to address the aforementioned problems mentioned above by developing alternative models to reduce ischemic spinal cord injury. The methods disclosed herein address these and other needs.

### SUMMARY

The present disclosure provides a method of reducing blood flow to the spinal cord in a vertebrate animal to induce paralysis or ischemic stroke. The present invention also provides a method of screening for therapeutic agents to be used in treating a central nervous system injury. The present disclosure provides an engineered vertebrate comprising reduced blood flow at the spinal cord to induce paralysis or ischemic stroke.

In one aspect, disclosed herein is a method of reducing blood flow to a segment of spinal cord in a non-human vertebrate animal, the method comprising dissecting a first intercostal space to expose a first group of intercostal arteries, keeping a second intercostal space intact, dissecting a third intercostal space to expose a second group of intercostal arteries, ligating the first group of intercostal arteries, and ligating the second group of intercostal arteries, thereby reducing blood flow to a segment of spinal cord, wherein the method comprises ligating five intercostal arteries.

In one embodiment, the first intercostal space and the third intercostal space are not adjacent to one another. In another embodiment, the second intercostal space lies between the first intercostal space and the third intercostal space. In one embodiment, the first group of intercostal arteries comprises an 8^{th}, a 9^{th}, and a 10^{th} intercostal artery. In another embodiment, the second group of intercostal arteries comprises a 6^{th} and a 7^{th} intercostal artery. In one embodiment, the 8^{th}, the 9^{th}, and the 10^{th} intercostal arteries are each ligated twice. In another embodiment, the 6^{th} and 7^{th} intercostal arteries are each ligated once.

In one embodiment, ligating any combination of the first and second group of intercostal arteries induces ischemic spinal cord injury in the vertebrate animal, wherein a more inferior intercostal artery is ligated first, and a more superior intercostal artery is ligated last. In another embodiment, ligating any combination of the first and second group of intercostal arteries mimics the effects of an ischemic brain injury in the vertebrate animal, wherein a more inferior intercostal artery is ligated first, and a more superior intercostal artery is ligated last.

In one embodiment, the first group of intercostal arteries and the second group of intercostal arteries branch from a descending aortic artery. In one embodiment, the non-human vertebrate is a rodent. In another embodiment, the rodent is a mouse.

In one aspect, disclosed herein is a method of screening for a therapeutic agent for treating a central nervous system (CNS) injury, the method comprising reducing blood flow to a segment of spinal cord in a non-human vertebrate to induce the CNS injury, wherein a first group intercostal arteries of the non-human vertebrate animal are ligated twice and wherein a second group of intercostal arteries are ligated once, administering at least one therapeutic agent to the non-human vertebrate, and selecting one or more therapeutic agents that increases CNS functions relative to an untreated control with the CNS injury.

In one embodiment, the central nervous system injury is an ischemic brain injury or an ischemic spinal cord injury.

In one embodiment, the first group of intercostal arteries comprises an 8^{th}, a 9^{th}, and a 10^{th} intercostal artery. In another embodiment, the second group of intercostal arteries comprises a 6^{th} and 7^{th} intercostal artery. In another embodiment, the method comprises ligating five intercostal arteries.

In one embodiment, the segment of spinal cord is a thoracic segment.

In one embodiment, the therapeutic agent comprises an analgesic, an anticonvulsant, an antivertigo, an anxiolytics, a sedative, an antibiotic, a CNS stimulant, a hypnotic, a muscle relaxant, or combinations thereof.

In one aspect, disclosed herein is an engineered non-human vertebrate animal comprising reduced blood flow to a segment of spinal cord, wherein a first group intercostal arteries are ligated twice and wherein a second group of intercostal arteries are ligated once wherein the non-human vertebrate is a model for ischemic spinal cord injury or ischemic brain injury, and wherein the non-human vertebrate animal comprises a hindlimb paralysis.

In one embodiment, the first group of intercostal arteries comprises an 8^{th}, a 9^{th}, and a 10^{th} intercostal artery. In another embodiment, the second group of intercostal arteries comprises a 6^{th} and 7^{th} intercostal artery. In another embodiment, the segment of spinal cord is a thoracic segment. In another embodiment, the vertebrate animal has 5 ligated intercostal arteries.

In some embodiments, the non-human vertebrate is a mammal. In some embodiments, the non-human vertebrate is a rodent. In some embodiments, the non-human vertebrate is a mouse.

### BRIEF DESCRIPTION OF FIGS

The accompanying figures, which are incorporated in and constitute a part of this specification, illustrate several aspects described below.

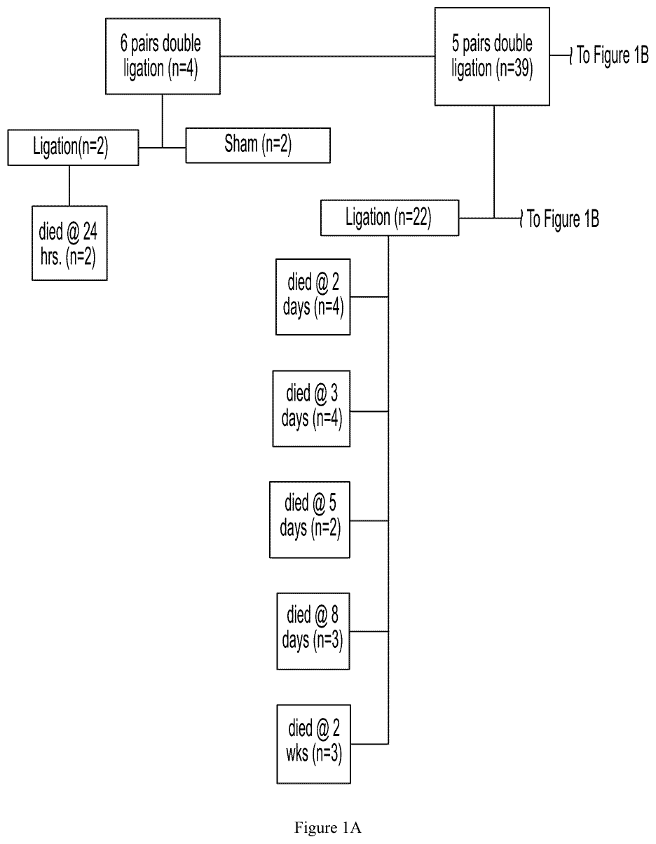

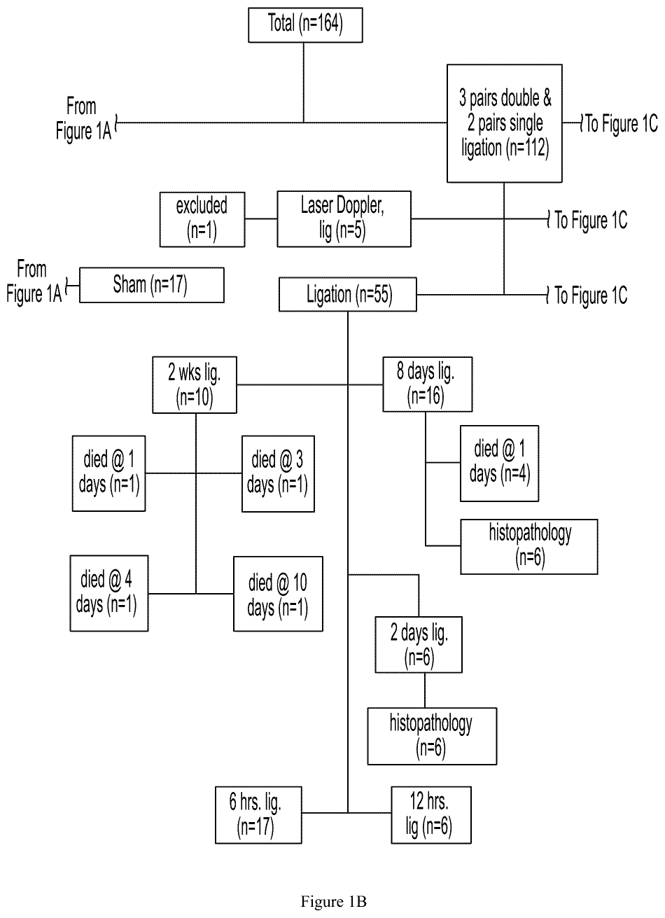

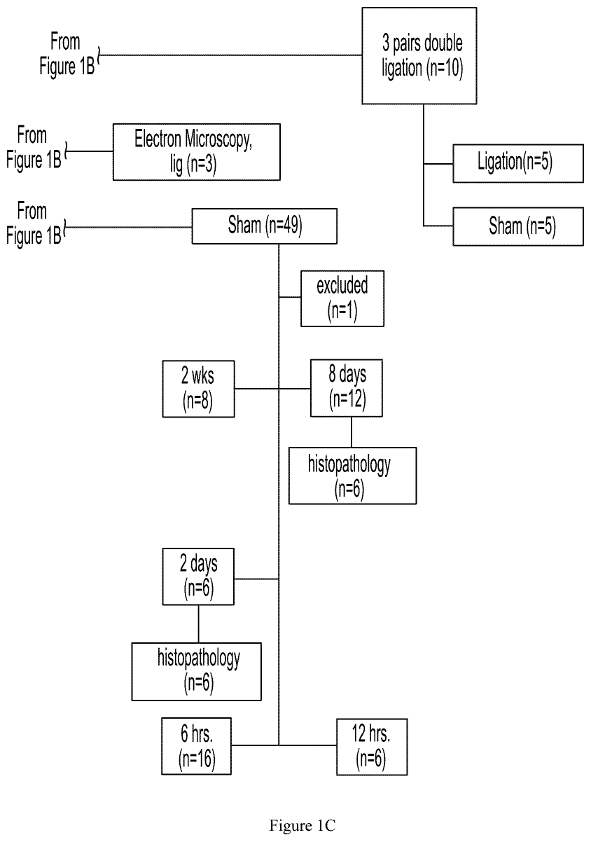

FIGS. 1A, 1B, and 1C show the animal tree of the total number of animals and their distribution throughout the study Animals that did not die were sacrificed at the end of the group time period.

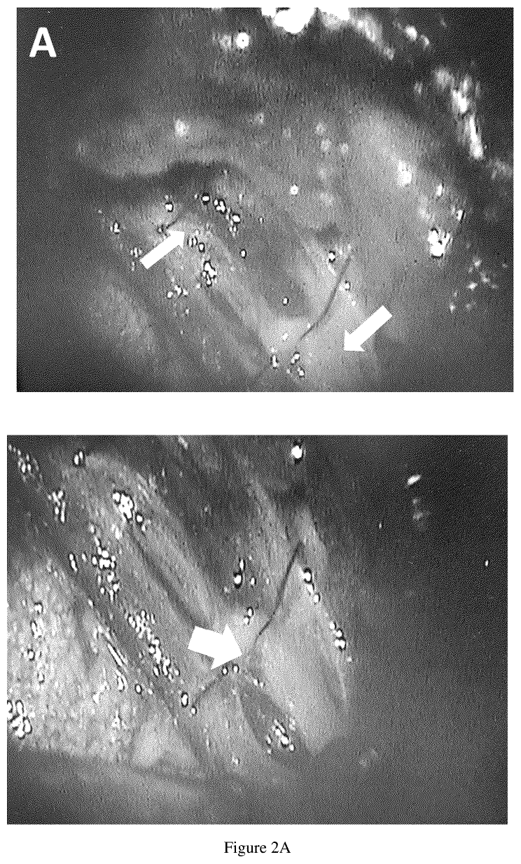

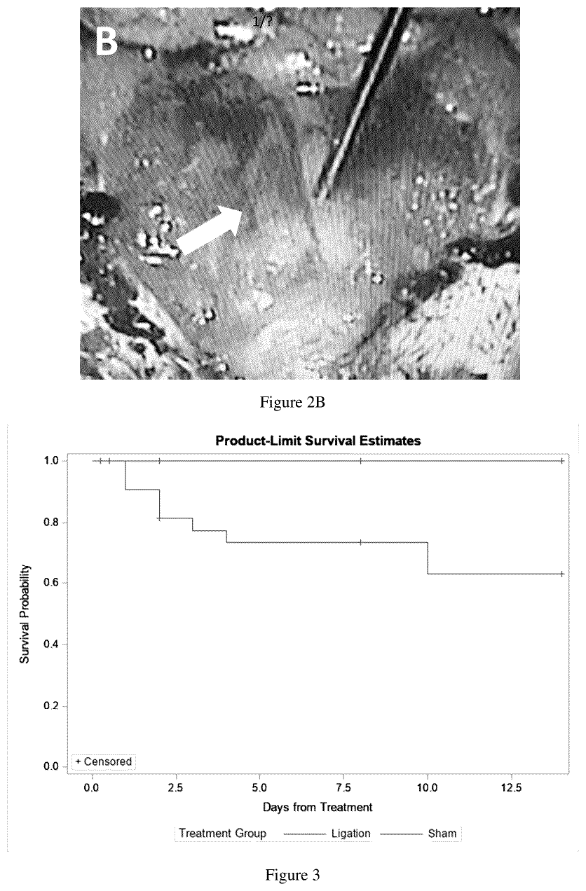

FIGS. 2A and 2B show the images of intercostal vessel ligation and laser doppler probe placement. FIG. 2A shows the intercostal vessel ligation after injecting with Evans blue dye. Arrows indicate the ligature sites. FIG. 2B shows the laser doppler probe measuring blood flow directly from the exposed mouse spinal cord. Arrow indicates placement of the probe over the spinal cord.

FIG. 3 shows the overall survival comparison between the ligation group (blue; n=55) and the sham group (red; n=48) groups over a two-week period using a Kaplan-Meier survival plot and Log rank test (Log-Rank test p-value=0.0040).

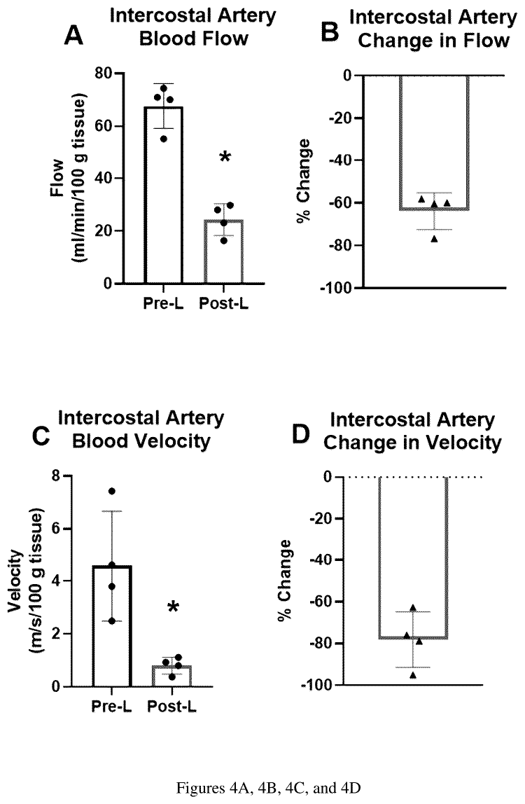

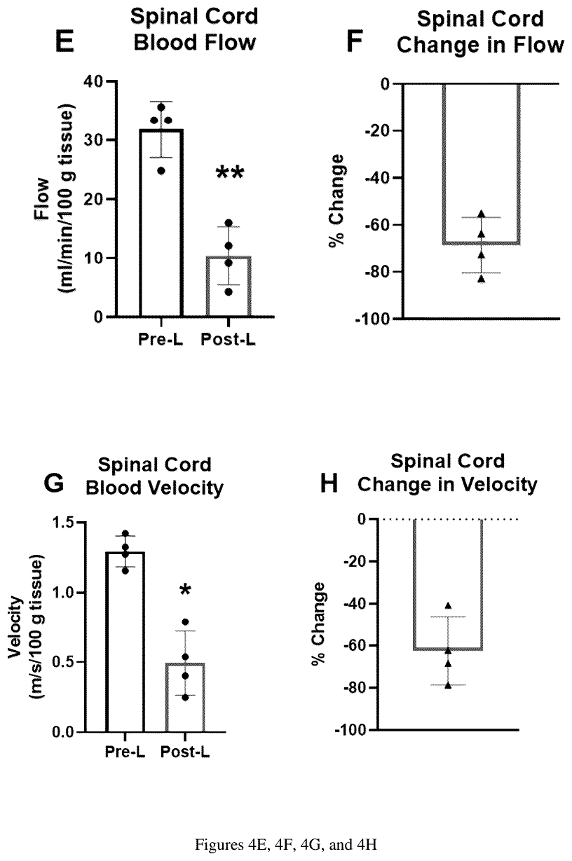

FIGS. 4A, 4B, 4C, 4D, 4E, 4F, 4G, and 4H show the localized blood hemodynamics measured by Laser Doppler tissue perfusion pre- and post-arterial ligation for intercostal artery and spinal cord blood flow (FIGS. 4A and 4E) with calculated % change in flow from pre- to post-ligation (FIGS. 4B and 4F); intercostal artery and spinal cord blood velocity (FIGS. 4C and 4G) with calculated % change in flow from pre- to post-ligation (FIGS. 4D and 4H). Pre-L=Pre-ligation, Post-L=Post-ligation. Data presented as mean±S.D. and analyzed by paired T-test. *p<0.05.** p<0.0001.

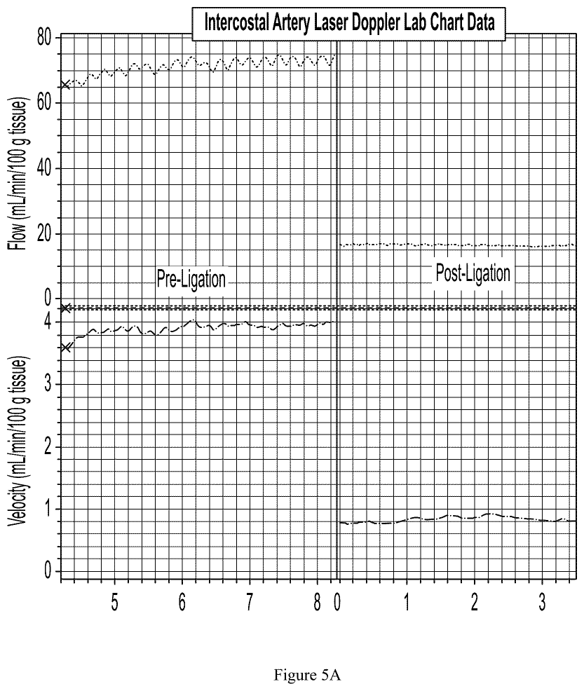

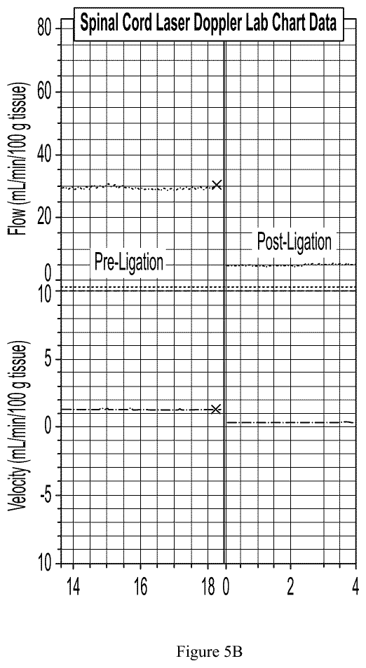

FIGS. 5A, 5B, and 5C show the LabChart data from Laser Doppler Analysis of Intercostal Artery and Spinal Cord blood flow. FIG. 5A shows the mouse 5 intercostal artery change in blood flow (red) and velocity (green) pre- and post-aortic ligation. FIG. 5B shows the mouse 4 spinal cord flow (red) and velocity (green) pre- and post-intercostal ligation. Notice the marked drop in spinal cord blood flow. FIG. 5C shows the mouse 2 spinal cord change in blood flow (red) and velocity (green) pre- and post-intercostal ligation. The drop in spinal cord blood flow is noticeably less than that in Mouse 4.

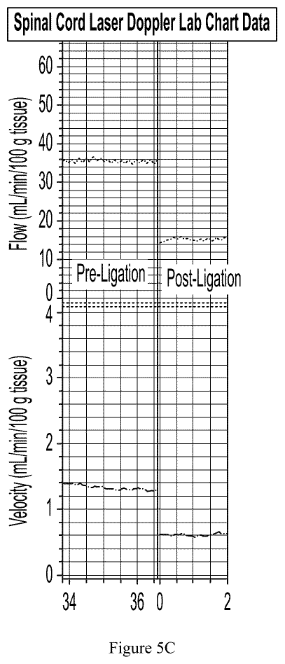

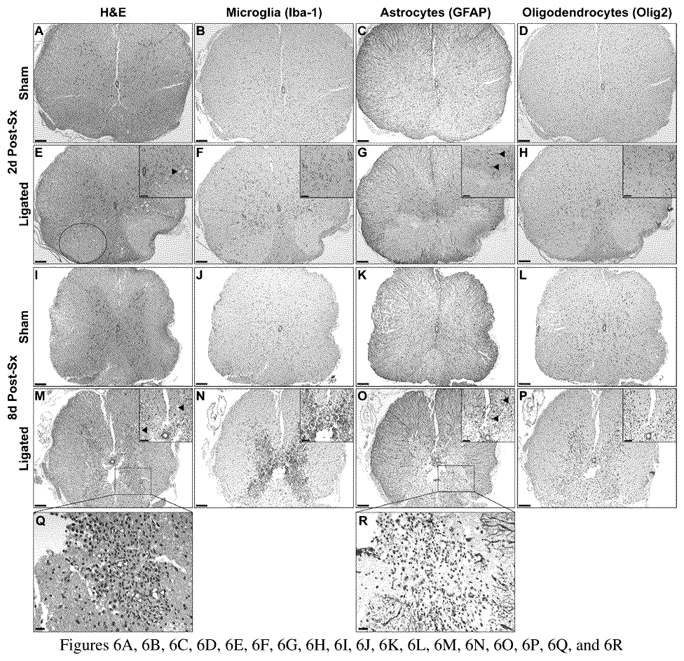

FIGS. 6A, 6B, 6C, 6D, 6E, 6F, 6G, 6H, 6I, 6J, 6K, 6L, 6M, 6N, 6O, 6P, 6Q, and 6R show that the ligation induces changes in the grey and white matter of the spinal cord at 2- and 8-days post-surgery. FIGS. 6A, 6B, 6C, 6D, 6E, 6F, 6G, and 6H show the mild histologic lesions are evident in the spinal cord at 2 days post-ligation compared to sham surgery. Hypocellularity in the dorsal horns (circle) is accompanied by multifocal swollen axon cylinders (spheroids, filled arrows) in adjacent white matter spinal tracts and hypercellularity in adjacent ventral grey matter. There are increased Iba-1+ microglia including large, activated microglia near the margins of hypocellular and hypercellular grey matter. Numerous large GFAP+ astrocytes are present in the dorsal horn grey matter around the central canal. There is a slight increase in Olig2+ oligodendrocytes in the grey matter and ventral white matter. FIGS. 6I, 6J, 6K, 6L, 6M, 6N, 6O, and 6P show that there are more severe lesions affecting most of the grey matter that are present at 8 days post-ligation compared to cords subjected to sham surgery. Grey matter in ligated mice is hypercellular with decreased neurons, increased glia, and multifocal proliferative small caliber blood vessels (FIG. 6Q, open arrows). Surrounding white matter tracts contain numerous swollen axon cylinders (spheroids, arrows). Grey matter contains numerous Iba-1+ microglia that frequently exhibit large, round, activated morphology. Activated, GFAP+ astrocytes in the grey matter and surrounding white matter (arrows) with the exception of areas containing proliferative blood vessels, where there are few GFAP+ astrocytes (FIG. 6R). Increased Olig2+ oligodendrocytes in the spinal grey matter of ligated mice. Scale bars in FIGS. 6A-6P=100 μm, taken at 100× total magnification. Insets in FIGS. 6E-6H and FIGS. 6M-6P taken at a total magnification of 200× with scale bars=50 μm. Q and R taken at a total magnification of 400× with scale bars=20 μm.

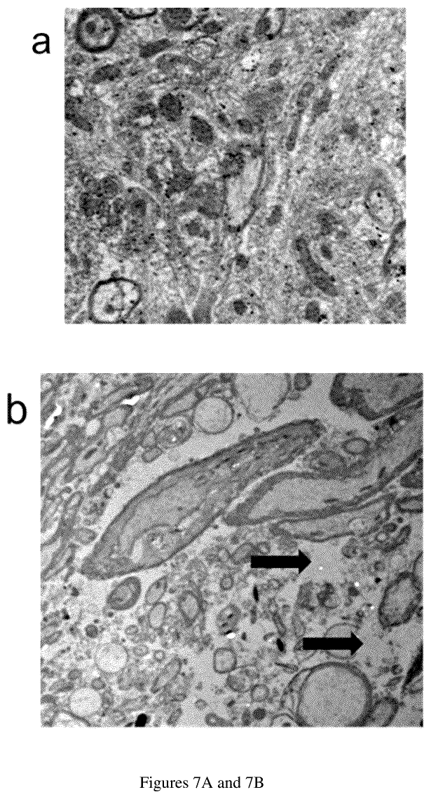

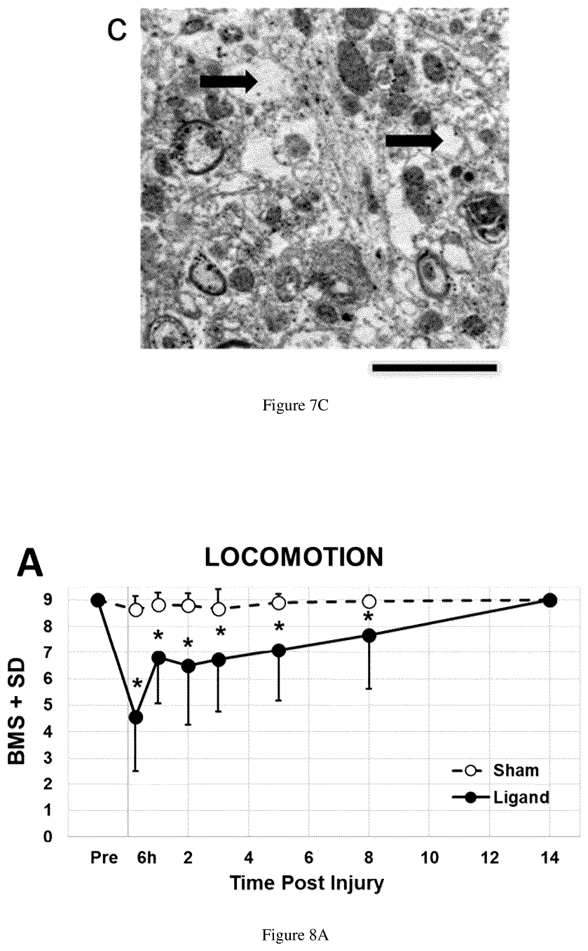

FIGS. 7A, 7B, and 7C show the transmission electron microscopy of sections from ischemia-induced mouse spinal cord segments showing the degrees of damage after ischemia. Ruptured cell membranes and an increase in extracellular space (arrowheads) are visible 24 hours after ischemia, and at 48 hours after ischemia, less extracellular space is visible, but more cellular damage, i.e., ruptured membranes, are visible, and no ischemic damage is present in the control. Scale bar: 1 um.

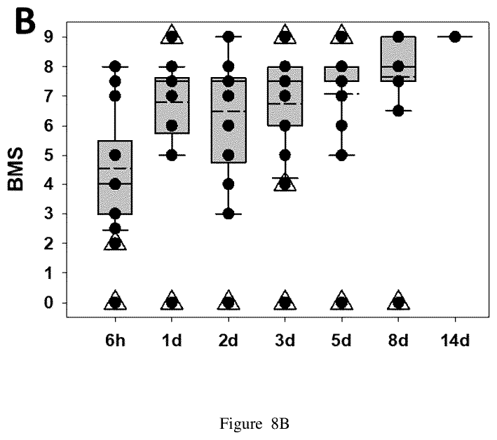

FIGS. 8A and 8B shows the Basso Mouse Scale Locomotor Scores show improvement through time and wide variability. FIG. 8A shows the average Basso Mouse Scale locomotor scores for the sham and ligation groups averaged across all animals (sham, n=48; ligation, n=55) tested at each timepoint. The groups are significantly different (*; p<0.01) through 8d. (Repeated measures ANOVA with Tukey's Post Hoc). FIG. 8B shows the wide variability in behavioral performance displayed in a box plot, showing both that mice scores are not tightly bunched at the mean. Black circles are individual values. Box edges show 25^{th} and 75^{th} percentile range, and whiskers show 10^{th} and 90^{th} percentile range. Points beyond that are shown in triangles and are designated as outliers. Dashed line is mean, and solid line is the median.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising,” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a,” “an,” “the,” include plural referents unless the context clearly dictates otherwise.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

An “increase” can refer to any change that results in larger amount of a symptom, disease, composition, condition, or activity. A substance is also understood to increase the genetic output of a gene when the genetic output of the gene product with the substance is more relative to the output of the gene product without the substance. Also, for example, an increase can be a change in the symptoms of a disorder such that the symptoms are more than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

The terms “prevent,” “preventing,” “prevention,” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

By “reduce” or other forms of the word, such as “reducing” or “reduction,” means lowering of an event or characteristic (e.g., ischemia). It is understood that this is typically in relation to some standard or expected value, in other words it is relative, but that it is not always necessary for the standard or relative value to be referred to. For example, “reduces ischemia” means reducing limited oxygen flow or blood flow relative to a standard or a control.

A “control” is an alternative subject or sample used in an experiment for comparison purpose. A control can be “positive” or “negative.” For example, where the purpose of the experiment is to determine a correlation of an altered expression level of a proto-oncogene with a particular type of cancer, it is generally preferable to use a positive control (a subject or a sample from a subject, carrying such alteration and exhibiting syndromes characteristic of that disease), and a negative control (a subject or a sample from a subject lacking the clinical syndrome of that disease).

As used herein, by a “subject” means an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient.

A vertebrate is an animal of a large group distinguished by the possession of a backbone or spinal column, including mammals, birds, reptiles, amphibians, and fish. Specifically contemplated are mammals in the family Muridae (rodents), such as mice, rats, hamsters, and gerbils.

A “central nervous system injury,” also known as traumatic injuries of the central nervous system (CNS), refers to any injury to the brain, spinal cord, or both. These injuries can result from, but are not limited to automobile incidents, sports injuries or falls, stroke, ruptured brain aneurysm, lack of oxygen, gunshots, or an explosive blast.

A “therapeutic agent” or “therapeutic” refers to a drug, protein, peptide, gene, compound, composition, or pharmaceutically active ingredient intended to treat or mitigate a disease, condition, or disease, such as central nervous system injuries.

As used herein, “vascular growth” refers to the process of the growth and branching of vessels of the body, such as blood vessels and lymphatic vessels. Proteins and peptides, such as growth factors, cell membrane receptors/proteins, and transcription factors are expressed to promote growth of vessels.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the invention may be applied preventively, prophylactically, palliatively, or remedially. Treatments are administered to a subject prior to onset (e.g., before obvious signs of ischemia), during early onset (e.g., upon initial signs and symptoms of ischemia), or after an established development of ischemia.

“Phenotype” refers to the set of observable characteristics or traits of an organism. This term covers an organism's morphology, or physical form and structure, its developmental processes, its biochemical and physiological properties, its behavior, and the products of behavior. As used herein, the phenotypes of ligating the intercostal arteries are mild, moderate, and severe paralysis.

“Degenerating tissue” or “tissue degeneration” refers to the process by which tissue deteriorates and loses its functional ability due to traumatic injury, aging, and wear and tear. Tissue degeneration affects all tissues of the body, but the natural degenerative process particularly affects the vertebral disc and joints of the spine and other joints of the body (e.g., knee, elbow, hip, etc.).

As used herein, “monitoring” refers to the actions of observing and checking the progress or quality of a treatment or procedure over a period of time. “Monitoring” also refers to observing the course of a disease or condition, such as a cancer, over a period of time.

As used herein, the term “thoracic” or “thorax” refers to an area of the body between the neck and the abdomen containing the heart, lungs, and the major blood vessels branching including the aortic and intercostal arteries.

A “TEVAR,” also known as a thoracic endovascular aortic repair, refers to treating an aneurysm, or a weak balloon-like bulge, in the upper part of the aorta. During this repair, a stent graft, a metal tube covered in fabric, is placed into the aorta to reinforce the aneurysm, and preventing it from bursting. Further complications can arise from TEVAR where the spinal cord is injured leading to paralysis.

The “aortic artery,” or the “aorta,” is the main artery that carries blood away from the heart into the rest of the body. When blood leaves the heart through the aortic valve, it travels through the aorta, which makes a cane-shaped curve and branches out into other arteries bringing blood flow to other parts of the body. The descending aortic artery refers to the linear and longest portion of the aorta that branches off into smaller arteries to supply blood to other parts of the body.

An “intercostal artery” refers to any artery that supplies to blood to the areas between the ribs, also called the intercostal space. The intercostal arteries are divided into three main arteries: the anterior intercostal artery, the musculophrenic artery, and posterior intercostal arteries. The anterior intercostal artery branches into the first six intercostal arteries. The musculophrenic artery branches into the 7^{th}, 8^{th}, and 9^{th} intercostal arteries. The posterior intercostal artery branches into the 10^{th} and 11^{th} intercostal arteries. Each arteries supplies blood to their respective spaces (spaces 1-11) thereby supplying blood to the ribs, spinal cord, pericardium, diaphragm, and abdominal muscles.

An “intercostal space” refers to is anatomical space located between two rib bones. The intercostal spaces are also located in the thoracic region of the spinal column, or spinal cord. Each space is numbered based on the rib bone superior (above) to it. Each space comprises layers of muscles, nerves, and vasculature to move the ribs during breathing.

As used herein, the term “ligation” refers to a surgical or medical procedure, wherein a ligature or knot consisting of a piece of thread (suture) is tied around an anatomical structure, usually a blood vessel, to slow or prevent normal circulation of fluids, usually blood. Here double ligation or single ligation are performed to prevent blood flow. A double ligation means to place two ligatures or two knots around one blood vessel to prevent blood flow at two distinct points or ligating the vessels twice. A single ligation means to place one ligature or knot around a blood vessel to prevent blood flow at one distinct point or ligating the vessels once.

As used herein, the term “segment” refers to a smaller portion of a larger whole. For example, a segment of spinal cord, comprising a total of 33 vertebrae, could refer to the thoracic segment of the spinal cord comprising the 8^{th}-19^{th} vertebrae, or T1-T12.

As used herein, the term “exposed” refers to making a structure or object visible, or open to view.

As used herein, “paralysis” refers to loss of the ability to move some or all parts of the body. Here, paralysis results from lack of blood flow in the spinal cord leading to a spinal cord injury, wherein the nerves of the spinal cord cannot function properly. A state of paralysis can be hindlimb paralysis, occurring only in the legs; forelimb paralysis, occurring only in the arms; or whole body paralysis, occurring throughout the entire body.

As used herein, “ischemia” refers to a condition in which blood flow and oxygen are restricted or reduced in a part of the body. “Ischemic spinal cord injury” or ischemic spinal cord paralysis refer to the occurrence when blood flow to the spinal cord is blocked or reduced for a period of time. “Ischemic brain injury” refers to the occurrence when blood flow to the brain is blocked or reduced for a period of time. Lack of blood flow, nutrients, and oxygen to the spinal cord or brain can lead to damage to the spinal cord or brain, and paralysis.

A “stroke” or a “brain stroke” refers to a specific type of ischemic brain injury when blood flow to the brain is blocked or reduced to prevent oxygen and nutrients from reaching the brain. A stroke or brain stroke also occurs when a blood vessel bursts or ruptures in the brain. These occurrences can lead to brain damage, long-term disability, or lethality. An “ischemic brain stroke” is the specific occurrence of blood flow blockage in the brain leading to brain damage and lethality.

As used herein, the term “hypoperfusion” refers to a reduced amount of blood flow to an organ or tissue thereby limiting or preventing its function. Here, a spinal cord hypoperfusion, also known as spinal cord ischemia, refers to blocked blood flow to the spinal cord leading spinal cord injuries, including loss of motor and sensory function usually to the arms and/or legs of the body.

The term “intubation” refers to a medical process, usually performed before major surgical procedures, where a tube is inserted through a subject's mouth or nose, then down into the trachea. The purpose of this process is to keep the trachea open and deliver oxygen to the subject during the subsequent major surgery.

The terms “dissecting,” “dissection,” or “dissect” are used herein to refer to the process of cutting apart or separating tissue for the purpose of performing a surgery or study anatomy of an animal or plant.

An “incision” is used herein refers to a cut or opening made into a tissue of the body by a scalpel, scissor, or blade usually made for the purpose of a surgery.

An “inferior” position refers to an anatomical position that is away from the head or lower relative to a set starting position. For example, the feet are the most inferior part of the body, or the abdomen is inferior to the head.

An “superior” position refers to an anatomical position that is towards the head or above a relative starting position. For example, the head is the most superior part of the body, or the neck is superior to the abdomen.

An “anterior” position refers to an anatomical position that is front facing relative to a set starting position. For example, the nose is anterior to the ears.

A “posterior” position refers to an anatomical position that is rear facing relative to a set starting position. For example, the shoulder blades are on the posterior side of the body.

As used herein, “lateral” refers to an anatomical position that is away from the midline of the body. For example, fingers are lateral to the chest or ears are lateral to the nose.

As used herein, “proximal” or “medial” refers to an anatomical position that is near the midline of the body. For example, the heart is proximal or medial to the hands.

A “laser doppler tissue perfusion probe” is a monitoring instrument placed on a part of the body or a specific tissue to continuously measure blood flow, mass, and velocity. As used herein, blood flow, mass, and velocity measurements allow complete assessment of preventing or blocking normal vascular functions following ligation of blood vessels.

The Basso Mouse Scale, or BMS, is a 9-point scale that provides a gross indication of locomotor ability and determines the phases of locomotor recovery and features of locomotion. Herein, BMS is used to scale the degree of paralysis, or limited locomotion, to an animal following ligation of intercostal arteries and limiting blood flow to the spinal cord.

As used herein, “grey matter” refers to portions of the brain that contains mostly neuronal cell bodies. The grey matter includes regions of the brain involved in muscle control and sensory perception.

As used herein, “white matter” refers to portions of the brain that contains the fatty substance, or myelin, that surrounds neuronal fibers, or axons.

### Methods

Thoracic aortic aneurysm is a weakened and widened area in the aortic artery usually requiring surgical intervention. Traditional aortic aneurysm repair methods, such as open repair and thoracic endovascular aortic repair (TEVAR), commonly lead to ischemic spinal cord injury and paralysis. In light of efforts to prevent ischemic spinal cord injuries due to previously mentioned aortic aneurysm repair methods, there are limited animal models of ischemic spinal cord injury.

Several existing open repair models provide pathophysiological evidence of post-ischemia reperfusion spinal cord injury. However, they have limited utility due to the high cost and difficulty of replicating large animal models (e.g., pigs, dogs), high morbidity and mortality rates, and the inability to genetically manipulate the animal. Lang-Lazdunski et al. (2000) attempted to resolve these limitations with the first open repair-induced ischemic spinal cord injury mouse model. However, the challenging surgical approach, high postsurgical mortality, and limited survivability (less than one week) precluded universal utilization. Thus, there remains a need to develop alternative models to reduce ischemic spinal cord injury.

The present disclosure provides a method of reducing blood flow to the spinal cord in a vertebrate animal to induce ischemic spinal cord injury or ischemic brain injury. The present invention also provides a method of screen for therapeutic agents to be used in treating a central nervous system injury. The present invention provides an engineered vertebrate comprising reduced blood flow at the spinal cord to induce ischemic spinal cord injury or ischemic brain injury.

The aortic artery is the large cane-shaped vessel that delivers oxygen-rich blood from the heart to the rest of the body. The aorta initially ascends upward to provide branching vessels to support blood flow to the head. Then, the aorta descends downward, by way of the descending aortic artery, and further branches into smaller vessels to support blood circulation to the upper limbs, lower limbs, major organ systems including, but not limited to the gastrointestinal, reproductive, urinary, and other supporting tissues. However, dysfunction in the aorta can lead to various diseases, disorders, and complications. One such complication is a thoracic aortic aneurysm in which the aorta widens as it passes through the chest, or thoracic region, of the body. Efforts to repair thoracic aortic aneurysms often lead to ischemic spinal cord injury where surrounding blood supply, through the intercostal arteries, to the spinal cord is blocked. The spinal cord is a vital structure and link between the brain and the body, protected by bones called vertebrae or the spinal column. Limited or complete loss of blood flow to the spinal cord can lead to paralysis, or limited mobility of the lower and/or upper limbs. While limited or complete loss of blood flow to the brain can lead to a stroke. To date, it is unknown how to prevent spinal cord ischemia following thoracic aortic aneurysm repair, and thus there is a need for an animal model of ischemic spinal cord injury.

Thus in one aspect, disclosed herein is a method of reducing blood flow to a segment of spinal cord in a non-human vertebrate animal, the method comprising dissecting a first intercostal space to expose a first group of intercostal arteries, keeping a second intercostal space intact, dissecting a third intercostal space to expose a second group of intercostal arteries, ligating the first group of intercostal arteries, and ligating the second group of intercostal arteries, thereby reducing blood flow to a segment of spinal cord, wherein the method comprises ligating five intercostal arteries.

In one embodiment, the first intercostal space and the third intercostal space are not adjacent to one another. In another embodiment, the second intercostal space lies between the first intercostal space and the third intercostal space. In one embodiment, the first group of intercostal arteries comprises an 8^{th}, a 9^{th}, and a 10^{th} intercostal artery. In another embodiment, the second group of intercostal arteries comprises a 6^{th} and a 7^{th} intercostal artery. In one embodiment, the 8^{th}, the 9^{th}, and the 10^{th} intercostal arteries are each ligated twice. In another embodiment, the 6^{th} and 7^{th} intercostal arteries are each ligated once. In some embodiments, the first group of intercostal arteries comprises ligating any combination of the 6^{th}, the 7^{th}, the 8^{th}, the 9^{th}, and the 10^{th} intercostal arteries. In some embodiments, the second group of intercostal arteries comprises ligating any combination of the 6^{th}, the 7^{th}, the 8^{th}, the 9^{th}, and the 10^{th} intercostal arteries.

In one embodiment, ligating any combination of the first and second group of intercostal arteries induces ischemic spinal cord injury in the vertebrate animal, wherein a more inferior intercostal artery is ligated first, and a more superior intercostal artery is ligated last. In some embodiments, ligating any combination of the first and second group of intercostal arteries mimics the effects of an ischemic brain injury in the vertebrate animal, wherein a more inferior intercostal artery is ligated first, and a more superior intercostal artery is ligated last.

In one embodiment, the first group of intercostal arteries and the second group of intercostal arteries branch from a descending aortic artery. In one embodiment, the vertebrate animal is a rodent. In another embodiment, the vertebrate animal is a mouse. In some embodiments, the vertebrate animal is a rat, hamster, or guinea pig. In some embodiments, the vertebrate is a dog, cat, rabbit, pig, sheep, cow, horse, frog, or fish. In some embodiments, the vertebrate is a non-human primate. In some embodiments, the vertebrate is a monkey or an ape.

In one aspect, disclosed herein is a method of screening for a therapeutic agent for treating a central nervous system (CNS) injury, the method comprising reducing blood flow to a segment of spinal cord in a non-human vertebrate to induce the CNS injury, wherein a first group intercostal arteries of the non-human vertebrate animal are ligated twice and wherein a second group of intercostal arteries are ligated once, administering at least one therapeutic agent to the non-human vertebrate, and selecting one or more therapeutic agents that increases CNS functions relative to an untreated control with the CNS injury.

In some embodiments, the central nervous system injury is an ischemic brain injury or an ischemic spinal cord injury.

In one embodiment, the first group of intercostal arteries comprises an 8^{th}, a 9^{th}, and a 10^{th} intercostal artery. In another embodiment, the second group of intercostal arteries comprises a 6^{th} and 7^{th} intercostal artery. In another embodiment, the method comprises ligating five intercostal arteries. In some embodiments, the first group of intercostal arteries comprises ligating any combination of the 6^{th}, the 7^{th}, the 8^{th}, the 9^{th}, and the 10^{th} intercostal arteries. In some embodiments, the second group of intercostal arteries comprises ligating any combination of the 6^{th}, the 7^{th}, the 8^{th}, the 9^{th}, and the 10^{th} intercostal arteries.

In one embodiment, the segment of spinal cord is a thoracic segment. In some embodiments, the segment of spinal cord is a cervical segment. In some embodiments, the segment of spinal cord is a lumbar segment. In some embodiments, the segment of spinal cord is an upper lumbar segment. In some embodiments, the segment of spinal cord is a sacral segment. In some embodiments, the segment of spinal cord is a coccygeal segment.

In one embodiment, the therapeutic agent comprises an analgesic, an anticonvulsant, an antivertigo, an anxiolytics, a sedative, an antibiotic, a CNS stimulant, a hypnotic, a muscle relaxant, or combinations thereof.

Exemplary analgesics include, but are not limited to acetaminophen, propofol, midazolam, etomidate, ketamine, barbiturates, dexmedetomidine, morphine, fentanyl, alfentanil, sulfenatil, codeine, and remifentanil. Exemplary anticonvulsants include, but are not limited to phenytoin, fos-phenytoin, levetiracetam, carbamazepine, phenobarbital, lamotrigine, gabapentin, oxcarbazepine, and valproate. Exemplary antivertigos (also referred to posttraumatic vertigo medication) include, but are not limited to betahistine dihydrochloride, meclizine, diphenhydramine, cyclizine, and promethazine. Exemplary anxiolytics include, but are not limited to benzodiazepine, diazepam, clonazepam, temazepam, lorazepam, midazolam, chlordiazepoxide, bromazepam, alprazolam, buspirone, and oxazepam. Exemplary sedatives include, but are not limited to barbiturates, benzodiazepines, nonbenzodiazepines hypnotics, antihistamines, opioids, and methaqualone, or derivatives thereof.

Exemplary antibiotics include, but are not limited to penicillins (including, but not limited to amoxicillin, clavulanate and amoxicillin, ampicillin, dicloxacillin, oxacillin, and penicillin V potassium), tetracyclins (including, but not limited to demeclocycline, doxycycline, eravacycline, minocycline, omadacycline, sarecycline, and tetracycline), cephalosporins (cefaclor, cefadroxil, cefdinir, cephalexin, cefprozil, cefepime, cefiderocol, cefotaxime, cefotetan, ceftaroline, cefazidme, ceftriaxone, and cefuroxime), quinolones (also referred to as fluoroquinolones include, but are not limited to ciprofloxacin, delafloxacin, levofloxacin, moxifloxacin, and gemifloxacin), lincomycins (including clindamycin and lincomycin), macrolides (including, but not limited to azithromycin, clarithromycin, erythromycin, and fidaxomicin (ketolide)), sulfonamides (including sulfamethoxazole and trimethoprim, and sulfasalazine), glycopeptides (including, but not limited to dalbavancin, oritavancin, telavancin, and vancomycin), aminoglycosides (including, but not limited to gentamicin, tobramycin, and amikacin), carbapenems (including, but not limited to imipenem and cilastatin, meropenem, and ertapenem), and topical antibiotics (including, but not limited to neomycin, bacitracin, polymyxin B, and praxomine) used alone or in combination.

Exemplary CNS stimulants include, but are not limited to amphetamines, armodafinil, atomoxetine, methylphenidate, modafinil, oxybate, pitolisant, and solriamfetol.

Exemplary hypnotics include, but are not limited to zolpidem, suvorexant, butabarital, lemborexant, quazepam, estazolam, flurazepam, triazolam, tasimelteon, eszopiclone, daridorexant, temazepam, ramelteon, secobarbital, doxepin, and zaleplon.

Exemplary muscle relaxants include, but are not limited to cyclobenzaprine, methocarbamol, diazepam, orphenadrine, baclofen, tizanidine, dantrolene, oxazepam, carisoprodol, metaxalone, and chlorzoxazone.

In one embodiment, the non-human vertebrate is a mammal. In some embodiments, the non-human vertebrate is a dog, cat, rabbit, pig, sheep, cow, horse, frog, or fish. In some embodiments, the non-human vertebrate is a non-human primate. In some embodiments, the non-human vertebrate is a monkey or an ape. In another embodiment, the non-human vertebrate is a rodent. In another embodiment, the non-human vertebrate is a mouse. In some embodiments, the non-human vertebrate is a rat, hamster, or guinea pig.

### Animal Models

The present invention provides an engineered non-human vertebrate comprising reduced blood flow at the spinal cord to induce ischemic spinal cord injury or ischemic brain injury.

In one aspect, disclosed herein is an engineered non-human vertebrate animal comprising reduced blood flow to a segment of spinal cord, wherein a first group intercostal arteries are ligated twice and wherein a second group of intercostal arteries are ligated once, wherein the non-human vertebrate is a model for ischemic spinal cord injury or ischemic brain injury, and wherein the non-human vertebrate animal comprises a hindlimb paralysis.

In one embodiment, the vertebrate is a model for ischemic spinal cord injury. In another embodiment, the vertebrate is a model for ischemic brain injury.

In one embodiment, the first group of intercostal arteries comprises an 8^{th}, a 9^{th}, and a 10^{th} intercostal artery. In another embodiment, the second group of intercostal arteries comprises a 6^{th} and 7^{th} intercostal artery. In another embodiment, the segment of spinal cord is a thoracic segment. In some embodiments, the segment of spinal cord is a cervical segment. In some embodiments, the segment of spinal cord is a lumbar segment. In some embodiments, the segment of spinal cord is an upper lumbar segment. In some embodiments, the segment of spinal cord is a sacral segment. In some embodiments, the segment of spinal cord is a coccygeal segment.

In one embodiment, the vertebrate animal has 5 ligated intercostal arteries. In some embodiments, the first group of intercostal arteries comprises ligating any combination of the 6^{th}, the 7^{th}, the 8^{th}, the 9^{th}, and the 10^{th} intercostal arteries. In some embodiments, the second group of intercostal arteries comprises ligating any combination of the 6^{th}, the 7^{th}, the 8^{th}, the 9^{th}, and the 10^{th} intercostal arteries. In another embodiment, the vertebrate animal has hindlimb paralysis.

In one embodiment, the vertebrate animal is a mammal. In some embodiments, the vertebrate animal is a dog, cat, rabbit, pig, sheep, cow, horse, frog, or fish. In some embodiments, the vertebrate animal is a non-human primate. In some embodiments, the vertebrate animal is a monkey or an ape. In another embodiment, the vertebrate animal is a rodent. In another embodiment, the vertebrate animal is a mouse. In some embodiments, the vertebrate animal is a rat, hamster, or guinea pig.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: A Mouse Model of Spinal Cord Hypoperfusion with Immediate Paralysis Caused by Endovascular Repair of Thoracic Aortic Aneurysm

A clinically relevant mouse model of thoracic endovascular aortic repair-induced ischemic spinal cord injury has been lacking since the procedure was first employed in 1991. It was thought that ligation of mouse intercostal arteries simulates thoracic endovascular aortic repair-induced ischemic spinal cord injury and behavioral deficit. A mouse model of thoracic endovascular aortic repair-induced spinal cord hypoperfusion was created by ligating five pairs of mouse intercostal vessels. This model represents reproducible spinal cord hypoperfusion causing spinal cord histopathological ischemic damage resulting in variable behavioral deficit and gradual improvement, mimicking the variability in radiological and clinical findings in human patients. This model permits the mechanistic study of the pathophysiology and molecular mechanisms underlying thoracic endovascular aortic repair-induced spinal cord hypoperfusion and proves useful as a preclinical tool for screening neuroprotective therapeutics.

### Methodology: Animals

Adult C57BL/6 mice (age range between 16-20 weeks for both males and females) were purchased from Jackson Laboratories (Bar Harbor, ME). The mice were randomly assigned to the sham and ligation groups in each of the pilot and final configurations: pilot using 3 double ligation (sham, n=5; ligation, n=5); pilot using 5 double ligation (sham, n=17; ligation, n=22); pilot using 6 double ligation (sham, n=2; ligation, n=2); final configuration using 3 double ligation with 2 single ligation (sham, n=48; ligation, n=55). In the final configuration, one sham mouse was excluded from the study due to hind limb injury unrelated to the surgery. FIG. 1 illustrates the experimental design with total number of animals and the detailed number of animals in each group. Neither body weight nor distribution of each sex differed significantly across the groups. All male mice (n=58) were >23 g, 31 (58.5%) of the female mice were ≤23 g and 22 (41.5%) were >23 g. Mice were blindly chosen for either the sham or ligation surgery using the procedure described infra in Randomization and Blinding.

All procedures used aseptic technique and all mice were housed in HEPA-filtered Bio-clean units. All procedures complied with the National Institute of Health Guide for the Care and Use of Laboratory Animals and were approved by The Ohio State University's Animal Care and Use Committee.

### Anesthesia Preparation:

The core temperature for all mice was maintained between 33.0-33.5° C. on a temperature-controlled surgical platform (World Precision Instruments, Sarasota, FL). Mice were anesthetized using 4% isoflurane in 100% 0 2 for induction, then maintained at 2% isoflurane (100 mL/minute O₂) throughout all surgical procedures. Two layers of disinfection, first with liquid surgical scrub and second with 70% ethanol were performed to clean the surgical site after shaving the hair. A lubricated mouse tracheal intubation cannula was inserted into the trachea (Hugo Sachs VK32) through the mouth after exposing the trachea with a vertical ventral midline incision in the neck extending slightly past the ear pinna. Then the larynx and trachea were exposed by retracting the overlaying fat, muscle, and submaxillary gland. A mouse ventilator [Hugo Sachs-Harvard Apparatus Minivent, Hollinston, MA: tidal/stroke volume=250 μL; rate=230 ventilations/min] was used for ventilation. The throat incision was closed with surgical glue and a single suture, and the tracheal cannula was fixed in place to the lip of the mouse with a surgical clip. Then the mouse was injected subcutaneously at the site of the incision with Bupivacaine (0.1 mg/mouse in 0.02 mL) and Gentamycin (0.1 mg/mouse in 0.1 mL). Five to 10 minutes later, the chest wall was opened.

To put animals in “relative hypothermia, the same core body temperature of 33° C.-33.5° C. was used to start because this is the same core body temperature and condition employed by Awad, et. Al (2010) while developing the open repair mouse model in 2010. Awad et. al (2021) found that this relative hypothermia reduced the high mortality rate, labored breathing, and seizure-like activities associated with the higher core body temperatures (33° C.-33.5° C.) used in the Lang-Lazdunski (2000) model. In addition, general anesthesia causes mild and sometimes moderate hypothermia, and this occurs under general anesthesia in both open and endovascular repair of aortic aneurysm. Therefore, to ensure animal welfare, avoid animal suffering and animal loss, and simulate the clinical setting, it was decided to use the same core body temperature used in the open model by Awad et. al. (2021).

### Surgical Procedure:

Intubated mice were placed in a right lateral position and the left forelimb was extended beneath the mandible and secured to the surgical platform with adhesive tape to expose the lateral thoracic cage beneath the left scapula. A small transverse incision was made underneath the left shoulder, then a blunt dissection of the subcutaneous fat to expose the underlying rib cage was performed. Using scissors, skin was cut longitudinally (rostral to caudal) to the lower costal cartilage, then the fat overlying the rib cage was bluntly dissected and moved to either side exposing the entire left thoracic cage. The intercostal muscles between the 8^{th} and 9^{th} ribs were cut with scissors, exposing the lateral pleura. With the incision wide open, the lower lobe of the lung was pushed out of the field using retractors while the skin and fat on each side were held away from the incision site using lateral hooks. The subcutaneous fat was kept moist throughout the procedure using normal saline. Using forceps while viewing through a 10× power microscope lens, the fully exposed descending aorta was bluntly dissected in a rostral to caudal direction while carefully exposing the intercostal arteries (8^{th} to 10^{th}). A second incision was made in the muscle between the 6^{th} and 7^{th} rib and retractors were used to hold it wide open and to push the lungs away from the field. While viewing through a 16× power lens, the exposed descending aorta was bluntly dissected a second time in a rostral to caudal direction to increase the exposure and visibility of the intercostal arteries (6^{th} through 10^{th}).

Continuing with the 16× power lens, the intercostal arteries were sutured (ligated) bilaterally using 9-0 nonabsorbable nylon sutures (ETHILON) starting with the lower-most vessel (10^{th} intercostal pair) and ending with the 6^{th} intercostal pair; the 8^{th}, 9^{th}, and 10^{th} artery pairs were double ligated, and the 6^{th} and 7^{th} pairs were single ligated. This configuration was chosen after conducting a pilot study where double ligating was first tried at the 11^{th}-6^{th} (in the six double-ligation group), the 10^{th}-6^{th} (in the five double ligation group), and the 10^{th}-8^{th} (in the three double ligation group). The rib cage and intercostal muscles were then sutured at the two incision sites using 6-0 polypropylene after absorbing any fluid or blood leak at the incision site. Finally, subcutaneous fat was put back after being moistened, and the skin was sutured using 5-0 polypropylene sutures. FIG. 2A illustrates an intercostal ligation after intracardiac injection of 200 μL Evans blue to demonstrate the aorta and its branches. Sham control mice underwent the exact same surgical procedure without ligation of the intercostal arteries.

The intercostal arteries in mice have a microscopic diameter of approximately 0.15 mm and are very delicate. Any relatively rough manipulation will lead to injury of the vessel and sometimes complete avulsion of the vessel. Given this, ligation was performed gently, which, in turn, makes the “tie” loose and does not completely restrict blood flow. To overcome this problem, double ligation was employed (double tied), which resulted in a complete cessation of blood flow and produced the behavioral and histopathological results.

### Post-Surgical Care:

All animals recovered spontaneously from anesthesia within 10-20 minutes on the surgical platform while their core temperatures were maintained. The mice were extubated and placed into new clean cages maintained on a warmer set at 34.5° C. for two to five days. Bladder care by a gentle manual evacuation of the bladder was performed every 12 hours for the duration of the experiment to prevent urine retention and infection. Mice were given 0.5 mL 10% dextrose in water subcutaneously twice daily and were also given prophylactic antibiotics—Gentamycin (0.1 mg/mouse in 0.1 mL) and Baytril (0.2 mg/mouse in 0.2 ml) twice daily subcutaneously through the first seven days post-surgery. Mice were kept on a regular diet supplemented by a Stat high caloric diet (PRN Pharmaceutical, Pensacola, FL) throughout the study. Animal welfare, post-operative pain control, and humane endpoints were ensured Per the protocols established by the Animal Care and Use Committee at The Ohio State University.

Per the protocols established by the Animal Care and Use Committee at The Ohio State University (OSU) protocol all mice that underwent any surgical procedure were administered by pain killers intra and postoperatively as shown in Table 2 and as discussed below. Additionally, a pain assessment was performed on all mice that underwent any surgical procedure using the mouse grimace scale preoperative (Baseline). The assessment was performed every 6 hours during the day, and 12 hours during the nights after surgery for three days, then twice daily till the endpoint. Clinically, organ function damage was not expected because the ligation causes a different outcome than the aortic cross clamping “open repair” surgery where ischemia reperfusion injury affects all organs below the site of cross clamping. The ligation surgery causes hypoperfusion at the level of the intercostal vessels that have been ligated, which predominantly affects only the spinal cord.

If the animal experienced seizures, labored breathing, more than 10% loss of body weight, or if the animal developed pain that was not responding to a dose of Meloxicam or Buprenorphine, the animal was humanely sacrificed.

### Tissue Perfusion Measurements:

Tissue perfusion data was obtained from five male mice using a Transonic Type N24 needle probe connected to a BLF22 laser doppler system (Transonic Systems Inc., Ithaca, NY). Twenty-four hours before measuring tissue perfusion, the mice underwent dorsal laminectomy of T8-T10, and the skin was stapled, and the mice left to recover after an injection of Gentamycin (0.1 mg/mouse in 0.1 mL). The next day, the mice were anesthetized with 2% Isoflurane in 100% oxygen, the spinal cord was exposed, and the laser doppler probe was stabilized over a marked location on the spinal cord using a micromanipulator (FIG. 2B). Spinal cord perfusion measurements were obtained before ligation. Mice were then placed in a left lateral position, the chest was opened, and the intercostal arteries were surgically ligated (as discussed in Surgical Procedure).

Intercostal artery blood flow measurements were taken before and after ligation by applying the probe tip on the surface of the vessels. The mouse was then switched back to the prone position and the tip of the laser doppler probe was placed on the spinal cord at the same marked location to acquire post-ligation spinal cord tissue perfusion measurements. In accordance with the protocol approved by The Ohio State University Institutional Lab Animal Care and Use Committee, all mice were sacrificed at the end of the tissue perfusion measurement. Powerlab data acquisition system and LabChart software (ADInstruments Inc., Colorado Springs, USA) were used to record the output signals from the laser doppler system with output signals set to the tissue perfusion flow unit (mL/min/100 g tissue) and blood velocity unit (m/s/100 g tissue).

Several factors were addressed before use of the laser doppler tissue perfusion probe. The probe was immobilized during the entire measurement to ensure proper measurement of reflected light. This was ensured by surgical implantation of the probe via micromanipulator. A mark for doppler measurement location ensured consistent pre- and post-ligation probe placement. Care was taken to avoid arterial occlusion by the probe and duplicate readings were taken to ensure consistent measurement upon placement. Additionally, the same ambient room lighting was maintained during the experiment to prevent confounding the results.

Behavioral Assessment:

Bilateral hindlimb function was blindly assessed with the Basso Mouse Scale (BMS) for Locomotion at six hours, days one, two, three, five, eight and two weeks after injury (see infra-Randomization and Blinding for a discussion of the blind randomization procedure used for the behavioral assessment). The BMS, a 10-point scale (0-9) with operational definitions of key locomotor features, quantifies the rate and extent of functional impairments in mice with neurovascular injury.

The Basso Mouse Scale for Locomotion (BMS) is a semi-quantitative 10-point scale to measure functional performance from complete hindlimb paralysis (score 0) to normal walking (score 9) in mice (BMS ref). Two testers observe the behavior of the mouse in a large round enclosure for 4 minutes and assign a score based on operational definitions of locomotion ranging from ankle joint movements (scores 1-2), weight supported stepping (scores 4-5), forelimb-hindlimb coordination (scores 6-7), and trunk control (score 8). The locomotor score can predict the neuropathology of white matter sparing after spinal cord injury across a range of severities and multiple mouse strains.

The BMS scale has strong psychometrics (validity, reliability, sensitivity) and demonstrates good sensitivity in progressive neurovascular lesions like contusion and endovascular aneurysm models. A mixture of early time points and later assessments were designed to detect the onset of dysfunction and any lasting deficits. This time course aligns with human progression of dysfunction. An in-depth description of the BMS and its psychometrics is available.

### Tissue Harvesting:

Mice were anesthetized with 7.5 m/kg Ketamine and 3.75 ml/kg Xylazine and then transcardially perfused with 25 ml 0.1 M phosphate-buffered saline, followed by 50 ml of 10% Formalin (see infra-Randomization and Blinding for a discussion of the randomization procedure used to select mice for tissue harvesting). Spinal cords were removed and postfixed in 10% formalin for five days.

### Histopathology:

Spinal cords from an equal number of male and female mice subjected to sham surgery or surgical ligation were collected at 2 days (sham, n=6; ligated, n=6) and 8 days (sham, n=6; ligated, n=6). The Comparative Pathology and Digital Imaging Shared Resource (CPDISR) of The Ohio State University Comprehensive Cancer Center performed all pathology procedures. An experienced prosection technician trimmed spinal cords in the area of experimental interest (lower thoracic and upper lumbar segments where the hypoperfusion was expected) to identify regions of ischemia. Additional spinal cord tissue immediately proximal to the areas of expected ischemia was also trimmed and processed. Spinal cords were further post-fixed for 48 hours in 10% formalin. Tissues were routinely processed for histopathology on a Leica Peloris 3 Tissue Processor (Leica Biosystems, Buffalo Grove, IL) and embedded in paraffin. The spinal cords were sectioned at an approximate thickness of 4-5 micrometers to produce multiple cross sections per region of interest, and batch stained with hematoxylin and eosin (H&E) on a Leica ST5020 autostainer (Leica Biosystems, Buffalo Grove, IL) using a routine and quality-controlled protocol.

Immunohistochemistry to detect the presence of astrocytes positive for Glial Fibrillary Acidic Protein (GFAP, Dako Product #Z0334, Agilent, Santa Clara, CA; 1:5000=0.58 0 g/mL), microglia positive for Ionized Calcium Binding Adaptor Molecule 1 (Tha1, Novus Biologicals Product #NB100-1028, Centennial, CO; 1:1000=0.5 0 □g/mL), oligodendrocytes positive for Oligodendrocyte Transcription Factor (Olig2, Abcam Product #109186, Boston, MA; 1:400=0.325 □g/mL), and apoptotic cells and bodies positive for cleaved caspase 3 (Cell Signaling Technologies Product #9661, Danvers, MA; 1:180=0.26 μg/mL) was performed using a Lab Vision 360 Automatic Immunostainer (Thermo Scientific, Kalamazoo, MI) with optimized and quality controlled protocols specific to each primary antibody. All immunohistochemistry procedures follow optimized, validated protocols for paraffin embedded tissues with confirmed quality control for antibody specificity, including positive, negative, no primary, and isotype controls for all antibodies.

All slides were evaluated by an American College of Veterinary Pathologists board-certified comparative pathologist (Dr. Corps) using a Nikon Eclipse Ci-L Upright Microscope (Nikon Instruments, Inc., Melville, NY). Representative photomicrographs were taken using an 18 megapixel Olympus SC180 microscope-mounted digital camera and cellSens imaging software (Olympus Life Science, Center Valley, PA).

### Electron Microscopy:

Fresh spinal cord samples (n=3) were fixed for a minimum of two hours at room temperature using 2.5% glutaraldehyde in 0.1M phosphate buffer, pH 7.4 in a volume of fixative that was greater than ten times the total volume of the tissue sample being fixed. Samples were stained using a LYNX tissue processor (Electron Microscopy Sciences) according to the following protocol: rinsed three times for five minutes each in 0.1M phosphate buffer, stained for one hour in 1% osmium tetroxide in 0.1M phosphate buffer, rinsed three times in distilled water, stained in aqueous 1% uranyl acetate for one hour, rinsed three times in distilled water, then dehydrated in an ethanol series (50%, 70%, 80%, 95%, 95%, 100%, 100%, 100%) where each step from 50% to 95% was 10 minutes long and each 100% step was 15 minutes. Samples were then changed to acetone for ten minutes and the resin infiltration series began according to the following schedule: 1:1 acetone:resin mix without accelerant for one hour, 1:2 acetone:resin mix without accelerant for 1 hour, two changes of 100% resin mix with accelerant over two to six hours. The resin mix used was mixed according to the manufacturer's recommendations using the Eponate 12 kit (Ted Pella, Inc.).

Samples were transferred into silicone molds containing freshly made resin mix and cured for 24 to 48 hours at 60° C. Samples were then sectioned using an LC7 ultramicrotome (Leica) at 90 nm thickness and post-stained using aqueous 1% uranyl acetate for three minutes, rinsed in water, and stained with Reynold's lead citrate for two minutes. Samples were then rinsed in water a second time and imaged using an FEI Spirit G2 Biotwin transmission electron microscope (FEI).

### Randomization and Blinding:

All mice were subjected to preoperative behavioral assessment using BMS scale 5 days after arrival to OSU from Jackson laboratory. Any mouse with pre-operative abnormal behavior was not included. Then mice were randomly given numbers, and all mice were subjected to the same preoperative care.

Each surgery day, four mice underwent surgery as follows: one cage for females and one cage of males were taken to the surgery station. Then one mouse (of either sex) was randomly selected and then underwent surgery (ligation or sham), then another mouse of the opposite sex was subjected to the same surgery. This pattern was then repeated for two additional mice to receive the other type of surgery, such that four mice were operated upon on the same day under the same conditions to have two ligated mice (one male and one female) and two sham mice (one male and one female).

The behavioral assessments were also blindly randomized Prior to the performance of each behavioral assessment, all living mice to be tested that date (ligated and sham) at all timepoints (e.g., one day, three days, one week, etc.) were randomly assigned alphabetical letters (A, B, C, etc.). The behavioral assessments were performed and BMS scores assigned with the observers unaware of the status of each mouse. Once all assessments were complete at the end of the testing session, the blinding was resolved by matching each mouse's number to the randomized letter and the corresponding BMS score.

A blind randomization process was similarly followed when sacrificing the mice and for tissue harvesting and processing. Histopathology, electron microscopy, and statistical analysis were each performed by different team members that were blindly given the samples labeled as “group A” and “group B,” and were therefore unaware of which group was the sham or ligation.

### Statistical Analysis:

For surgical results, categorical variables are summarized as frequencies (percentage) and compared between groups using chi-squared tests or Fisher's exact tests where relevant. Continuous variables are summarized as means (standard deviation) and compared between groups using Student's independent t-tests. Mouse blood flow and velocity changes were compared between time points using paired t-tests. Overall survival was compared between study groups using a Kaplan-Meier survival plot and Log rank test. Hypothesis testing was 2-sided and p-values <0.05 were considered statistically significant. SAS version 9.4 (SAS Institute, Cary, NC) was used to conduct the statistical analysis.

For the Basso Mouse Scale data, the statistics could not be run in a single repeated measure, as animals were being deducted at multiple time points before the end of the study. A repeated measures ANOVA was performed for the interval of 6 h, 1 d, 2 d and a second analysis was performed for 3 d, 5 d, and 8 d with the repeated factor being time/days post-operative. Tukey's Post Hoc was used to compare the sham vs ligand at each time point. The graphs display all animals tested at each time point. The use of parametric statistics for the BMS and similar locomotor rating scales is supported by Abelson and Tukey and confirmed to be statistically appropriate for spinal cord injury.

For laser doppler tissue perfusion, all statistics were calculated by paired t-test. All data were calculated using GraphPad Prism 8.4.3. One mouse and all associated data were excluded (Mouse 3) from the study due to abnormal velocity detected with doppler ultrasound prior to ligation resulting in a positive outlier using the Grubbs Test.

No statistical power calculation was conducted prior to the study. The focus was the achievement of acceptable post-ligation mortality and paralysis rates. These were achieved with the final configuration of 3 pairs of double ligated and 2 pairs of single ligated intercostal arteries. Once this final configuration was chosen, the sample size was set to n=6 for each time point if the mice at that time point would only be subjected to behavioral assessment, and increased the n as needed if the mice at a given time point would also be examined subjected to another experiment, e.g., the histopathological study.

### Results:

### Survival Probability Post-Surgery:

In the pilot study, there was a significant difference in paralysis outcomes and mortality among the three ligation strategies at day 1. (Table 1). Increasing the number of arteries ligated and/or the number of ligations on each artery increased the severity of paralysis—but at a cost of survivability (Table 1). Double ligation of three pairs of intercostal arteries produced only mild paralysis (Basso Mouse Scale ≥7.5) at 24 hours with 0% mortality (n=5; p<0.001). Double ligation of six pairs of intercostal arteries caused 100% severe paralysis (Basso Mouse Scale≤4) at 24 hours with 100% mortality (n=2; p<0.001). Double ligation of five pairs of intercostal arteries produced 59.1% of mice with severe paralysis at 24 hours, but the mortality rate was still very high at 72.2% (n=22; p<0.001).

Based on these results, the final model used the configuration of double-ligation of three pairs of intercostals (8^{th}-10^{th}) and single ligation of two pairs of intercostals (6^{th} and 7^{th}) which resulted in three stratified groups of behavioral deficits, 9.4% severe paralysis, 37.5% moderate paralysis, and 53.1% mild paralysis. (n=55; p<0.001), and an overall mortality rate of 28.1% at day 1 (n=32) (Table 1).

Overall, considering both the pilot and final configurations, it was found that severely paralyzed mice (Basso Mouse Scale≤4) had the highest mortality rate (83.3%; n=18) compared to moderately paralyzed mice (4<Basso Mouse Scale<7.5) (33%; n=18) and mildly paralyzed mice (Basso Mouse Scale≥7.5) (24%; n=25) with p<0.001 (Table 1).

In the final model configuration, a significantly lower overall survival rate was also found for the ligation group (84%) compared to the sham group (100%) with p=0.0032 (n=48). No significant difference in survival probability between the ligation and sham groups occurred at 6-hours and 12-hours post-operation but was apparent at post-operative days 2, 8, and 14 (Table 3). FIG. 3 illustrates the overall survival comparison between ligation and sham groups over a two-week period. Mice that died in the first three days post-surgery expired due to respiratory difficulties. Mice that died after three days post-surgery expired due to excessive weight loss or infection.

### Tissue Perfusion Measurement:

Intercostal artery and spinal cord blood flow (ml/min/100 g tissue) and red blood cell velocity (m/s/100 g tissue) was measured in male C57BL/6 mice by laser doppler tissue perfusion (n=4). Baseline intercostal arteries blood flow (mean=67.66, SD=8.50, 95% CI=±8.34, p=<0.001) and velocity (mean=4.57, SD=2.09, 95% CI=±2.05, p=0.030) were higher than the thoracic spinal cord blood flow (mean=31.83, SD=4.79, 95% CI=±4.70, p=<0.001) and velocity (mean=1.30, SD=0.11, 95% CI=±0.11, p=<0.001) (Table 4). After intercostal arteries ligation, a significant and instantaneous drop in blood flow (% change) (mean=±63.81, SD=8.64, 95% CI=±8.47) and velocity (% change) (mean=−78.14, SD=13.28, 95% CI=±13.01) occurred in the arteries (FIGS. 4A, 4B, 4C, and 4D). The intercostal artery occlusion also caused significant instantaneous reduction of blood flow in the thoracic spinal cord (% change) (mean=−68.55, SD=11.91, 95% CI=±11.68) and an instantaneous reduction in blood flow velocity in the thoracic spinal cord (mean=−62.43, SD=16.10, 95% CI=±15.77) (FIGS. 4E, 4F, 4G, and 4H).

Ligation of five pairs of intercostal arteries induced spinal cord hypoperfusion resulting in a variable but significant drop in spinal cord blood flow. FIG. 4 and Table 4 show the mean blood flow and velocity in the ICAs and spinal cord pre- and post-ligation and the percent change in the intercostal and spinal cord blood flow and velocity pre-and post-ligation (n=4). FIG. 5A shows an example of the change in the intercostal artery blood flow and velocity pre- and post-ligation in one mouse. FIGS. 5B and 5C show examples of the change in the spinal cord blood flow and velocity pre- and post-ligation in two other mice. For example, one mouse experienced an 83.6% decrease in spinal cord blood flow post-ligation largely related to low spinal cord baseline blood flow levels (FIG. 5B, mouse 4). In contrast, another mouse had comparatively higher spinal cord baseline blood flow and experienced only a 55.1% decrease in spinal cord blood flow post-ligation (FIG. 5C, mouse 2).

### Histopathology:

Spinal cords from both male and female mice undergoing ligation were clearly distinguished from those undergoing sham surgical procedure without ligation. Within 2-day and 8-day ligation groups there was variability in the severity and distribution of lesions, and changes in the white matter were less pronounced than those in the grey matter unless necrosis was present. (FIG. 6).

Grey Matter Lesions: Prominent hypercellularity was present in both dorsal and ventral horn grey matter in ligated mice, particularly at 8 days post-ligation. Cells contributing to hypercellularity included large numbers of Iba-1+ microglia (FIG. 6N), GFAP+ astrocytes (FIG. 6O) and fewer Olig2+ oligodendrocytes (FIG. 6P). Microglia frequently had small, dense, round to oval, peripheralized nuclei with large, round to oval cytoplasmic surface area. Microglia in sham animals exhibited the expected morphology of thin arborizations and a small cell body. Astrocytes in and around affected grey matter had large, open oval nuclei with increased cytoplasm and plump, shorter cytoplasmic processes rather than the small soma and small, central round nuclei observed in spinal cords from mice subjected to sham procedure. Frequently, increased numbers of microglia and astrocytes exhibiting these respective morphological changes were present in viable tissue along the margins of foci of cell death and tissue loss. These changes were more prominent in spinal cords examined at 8 days post-ligation compared to 2 days post-ligation (FIGS. 6E, 6F, 6G, and 6H). Interestingly, in 3 spinal cords collected 2 days following ligation, the dorsal horns were markedly hypocellular with almost no neurons, microglia, astrocytes, or oligodendrocytes compared to sham spinal cords at the same time point (FIGS. 5E, 5F, 5G, and 5H). Unique in the ligated spinal cords examined at 8 days post-procedure were multifocal clusters of numerous small, proliferative capillaries with variable luminal diameter. These capillaries were frequently found in the dorsal horn in areas of marked hypercellularity and glial neuroinflammation (FIGS. 6Q and 6R).

White Matter Lesions: _Lesions in the white matter were highly variable in spinal cords examined at 2 days or 8 days post-ligation but were less pronounced than lesions in the grey matter. These changes included swollen axon cylinders (spheroids) in dilated myelin sheaths (FIGS. 6E and 6M) and occasional small foci of neuroinflammation (including increased microglia and astrocytes, FIGS. 6F and 6N, 6G and 6O) adjacent to blood vessels or foci of cell death. Spheroids were observed in all white matter tracts but often present in only 1-3 funiculi in an individual spinal cord. Variability in the size and severity of axon swelling was also common, with spheroids of varying size present in individual spinal cords at both time points.

Evaluation of cleaved caspase 3 immunohistochemistry at 2 and 8-days timepoints revealed rare, individualized cells with positive punctate cytoplasmic or defined nuclear staining. Positively labeled cells were rare, with a single positive cell present per spinal cord section (data not shown).

### Electron Microscopy:

In each sample, including the control, some fixation and expansion artifacts were present, therefore those criteria were not included in the assessment of ischemia-related damage. Rather, the relative severity of extracellular space and the presence of broken cell membranes relative to the control sample were relied upon as criteria for damage assessment. After intercostal ligation, extracellular space was substantially increased relative to the control 24-hour post-ischemia (FIGS. 7A and 7B). In contrast, by 48 hours, less extracellular space was present (FIG. 7C), but more cellular damage, i.e., ruptured membranes, was observed.

### Behavioral Assessment:

In all ligated mice, hind limb motor deficit of a variable degree was observed immediately after recovery from anesthesia. The 6-hour time point showed the lowest Basso Mouse Scale score. Over the two-week period, there was gradual increase of locomotor function of the mice that began 24 hours post-ligation and continuously improved from 3 d to 14 d (FIG. 8). The ligation group had significantly lower Basso Mouse Scale scores relative to sham through 8 days (p<0.01; FIG. 8A). After ligation, a large range of locomotor deficits occurred at each time point. The greatest variability occurred early after ligation such that at 6 hours Basso Mouse Scale scores ranged from 0 (no hind limb movement) to 8 (nearly normal locomotion) (FIG. 8B). By 14 days, the variability had narrowed substantially. Variability occurred both as underperforming and outperforming the group mean. A single mouse scored Basso Mouse Scale 0 at the initial test and showed no improvement over time, maintaining full hind limb paralysis until sacrificed at the 8-day time point. The relationship between severity of deficits and mortality was examined for high severity with Basso Mouse Scale scores≤4, moderate severity with Basso Mouse Scale scores>4 and <7.5, and mild severity with Basso Mouse Scale scores≥7.5. The Fisher's exact test showed that Basso Mouse Scale scores were significantly different across the four configurations (p<0.001) Table 1. For example, 100% of the 6 double ligation group (n=2), 59.1% of the 5 double ligation group (n=22), 9.4% of the 3 double and 2 single ligation group (n=32), and 0% of the 3 double ligation group (n=5) had Basso Mouse Scale scores<4 at day 1 with p<0.001.

### Discussion

Data obtained during ligation strategy selection demonstrates that the degree of paralysis is directly proportional to the number and degree of ligated intercostal arteries while survivability is inversely proportional to the number and degree of ligated intercostal arteries. The final configuration—3 double ligations and 2 single ligations of the intercostal arteries—caused clear motor deficits below the ligation level without high mortality for at least 14 days. Survival rate ranged between 100% in the 6-hour and 12-hour groups and 63% in the 2-week group. Together, the data shows that it is possible to moderate paralysis severity by varying the number of double-ligated arteries.

Four primary behavioral observations in the model demonstrate similarity with humans. First, the immediate paralysis occurring in this model is similar to the immediate paralysis occurring in humans after thoracic endovascular aortic repair and is in contrast to the delayed paralysis occurring in the open repair mouse model and humans. Importantly, the large Basso Mouse Scale score drop in ligated mice at 6 hours is non-attributable to anesthesia effects because the sham mice returned to normal or near normal behavior at this timepoint (FIG. 8A).

Second, wide behavioral deficit variability from severe to mild occurred in the mice in response to the same ligation procedure. (FIG. 8). These findings replicate the recent clinical profile in patients post-thoracic endovascular aortic repair.

Third, gradual paralysis improvement occurred throughout the two-week follow-up period with sustained improvement starting at day 3 (FIG. 8A). The variable degree of deficit and gradual improvement mimic the recovery process in humans who develop thoracic endovascular aortic repair-induced paralysis where mild paralysis was transient and improved with time while severe paralysis was permanent.

The deficit variability showing some improvement is attributed to extensive collateral circulation and adaptation to blockage. Etz et. al (2008 and 2011) proved that in humans, spinal cord perfusion pressure drops markedly but then recovers gradually during the first several hours after extensive segmental artery sacrifice. In the same study, it was shown that all but 1 patient, who had remarkably low postoperative spinal cord perfusion pressure and experienced paraparesis, regained normal spinal cord function. Etz et. al (2008 and 2011) also proved the existence of an extensive collateral network around the spinal cord in pigs. Griepp, et. al (2012) demonstrated that the collateral network around the pig spinal cord shows enlargement within 24 hours after extensive ligation of segmental arteries and that maximum collateral circulation expansion is achieved by the fifth day post-ligation and correlated these results to the finding of Etz et. al (2008 and 2011) in humans. All three studies demonstrate the similarity between spinal cord collaterals in humans and pigs as well as the similarity between humans and pigs in the temporal response to segmental artery ligation or sacrifice. In this model, similar behavioral/locomotor results were found in mice as those in humans with gradual improvement of motor function over the first few days in mildly and moderately paralyzed mice, and permanent loss of motor function in severely paralyzed mice. No study demonstrates the existence of collateral circulation around the spinal cord in mice. However, the similar dysfunctional findings in mice and the studies of humans and pigs leads to the conclusion that anatomical similarities—i.e., a collateral network around the spinal cord that gradually improves after intercostal artery ligation—should also exist in mice.

A critical foundation of this model included restricted spinal cord blood flow confirmation. Laser doppler measurement of spinal cord blood flow was extended from rats to mice to collect in vivo blood flow measurements from the exposed spinal cord and intercostal arteries. The observed percent change variability in spinal cord blood flow among ligated mice is explained by the existence of collaterals around the spinal cord and the degree of patency of these collaterals that varies among the ligated mice. Further, the degree of the collateral's patency and the pre-ligation baseline spinal cord blood flow variability explain the variability in the post-ligation percent decrease in spinal cord blood flow, which explains the spinal cord tissue damage variability and the resultant behavioral paresis among the ligated mice. It is therefore postulated that baseline spinal cord blood flow evidences the extent of collateral network development around the spinal cord—where a relatively low baseline blood flow demonstrates a less developed network, and a comparatively higher baseline blood flow demonstrates a more extensively developed network.

Thus, mice with more developed and patent collaterals associated with a lesser drop in blood flow, mild behavioral deficit, and progressive recovery over time. In contrast, mice with less developed and patent collaterals associated with a marked drop in blood flow, severe behavioral deficit Therefore, the pre-thoracic endovascular aortic repair degree of patency and development of collaterals around the spinal cord predict post-procedure paralysis severity and permanence, which proves to be important clinical screening characteristics.

Fourth, severely paralyzed mice were found to have the highest mortality rate (83.3%; n=18) compared to moderately paralyzed mice (33%; n=18) and mildly paralyzed mice (24%; n=25) with p<0.001. This relationship between spinal cord injury development and mortality aligns with 2020 national incidence and mortality data reported for thoracic endovascular aortic repair. Patients who developed reversible spinal cord injury symptoms had significantly worse 1-year survival rates than those without spinal cord injury (transient spinal cord, 80% [95% CI, 73%-87%]; no spinal cord injury, 87% [95% CI, 86%-88%]; log-rank p=0.1) but significantly better survival than those who developed permanent spinal cord injury symptoms (permanent spinal cord injury, 54% [95% CI, 47%-61%]; transient spinal cord injury, 80% [95% CI, 73%-87%]; p<0.0001).

Histopathological spinal cord changes from ligated mice also reflect ischemic change, severity variability, and precise localization of lesions. Apparent activation of astrocytes and microglia occurred post-ligation based on classic morphological changes in other sterile injury models. A more modest increase in Olig2+ oligodendrocytes was present in SCs examined 8 days post-ligation compared to sham control spinal cords, but no appreciable change in Olig2+ cells was present in spinal cords examined 2 days post-ligation (FIG. 6). Electron microscopic images differentiated abnormal cellular processes—broken cellular membranes and diffuse tissue damage after ligation. This supports the conclusion that the ligated animal's behavioral deficit is due to spinal cord ischemic damage and is not a mere side effect of the surgical approach.

Despite the lesion variability, a pattern of changes showing a temporal continuum emerged. In the grey matter of spinal cords at 2 days post-ligation, there is a paucity of cells, particularly in the dorsal horn but also extending multifocally into the ventral horns. These areas are surrounded by activated microglia and astrocytes and are multifocally accompanied by white matter lesions including histological evidence of axonal injury. In spinal cords at 8 days post-ligation, there is marked gliosis diffusely in the grey matter and in multifocal perivascular regions in the white matter composed predominantly of reactive microglia and astrocytes. This is frequently accompanied by proliferation of small capillaries (FIG. 6). A single spinal cord was characterized by loss of architecture and paucity of cells with replacement by cellular debris in approximately 60% of the examined section. This spinal cord corresponded to severe paralysis and Basso Mouse Scale score of zero at 8 days post-procedure. Of interest is the lack of cleaved caspase 3 immunolabeling. It is possible that apoptosis is a component of the model but is missed via immunohistochemistry at the examined time points or based on the immunohistochemistry findings at 2 days, apoptosis does not represent the predominant mechanism of cell death in this ischemia model.

These spatially variable lesions are consistent with MRI findings in humans with thoracic endovascular aortic repair-induced spinal cord injury that showed variability in location of the lesion in the spinal cord across the anterior gray matter and anterior portion of posterior white column, central cord lesion, and posterior gray matter, and posterior white column. This difference in the location and extent of infarction area can be explained by collateral conception. The drop in blood flow in the anterior spinal artery caused by ligation is partially compensated by flow from the collaterals in and around the spinal cord. The area of the critical drop in tissue perfusion within the spinal cord varies among ligated mice due to the variability in degree of collateral development and patency. Collateral conception together with ischemia-induced inflammation can explain the proliferation of small capillaries at the infarct area margins.

Electron microscopic examination provided another proof of ischemic spinal cord tissue damage. Because hypoperfusion-induced tissue damage results in tissue that contains abnormal (swollen or shrunken processes) or a diffuse type of damage not limited to cracking or breaks in the sample, this type of damage was not differentiated from the mechanically based cracking damage (FIG. 7). Abnormal cellular processes, broken cellular membranes, and diffuse tissue damage was more prevalent in the ligated samples. These findings further support the conclusion that the ligated animal's behavioral deficit is caused by spinal cord ischemic damage and is unrelated to the surgical approach.

### Conclusion:

Here the first mouse model with confirmed spinal cord hypoperfusion due to ligation of the intercostal arteries is presented reproducing the immediate onset of paralysis and mimics the variability in severity and reversibility of paralysis and paresis in human patients after thoracic endovascular aortic repair. This model also reproduces the thoracic endovascular aortic repair-induced spinal cord lesion histological topography observed in patients via MRI. These characteristics present a preclinical mouse model of ischemic spinal cord injury that holds good translational value and permit the discovery of neuroprotective drugs and therapeutics to prevent or treat ischemic spinal cord injury following thoracic endovascular aortic repair—a necessary step in the study and prevention of thoracic endovascular aortic repair-induced neurological deficits.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Comparison of severity of paralysis at day 1 based on Basso Mouse Scale (BMS) score between
ligation groups. Severe paralysis (BMS ≤4), moderate severity paralysis (BMS >4 & <7.5),
and mild severity paralysis (BMS ≥7.5) at 24 hours. Also shown are the mortality rates based
on severity of paralysis for all ligated mice and comparison of mortality by ligation group.

| | | | | | 3 double | | | | | | | |
|---|---|---|---|---|---|---|---|---|---|---|---|---|
| | 6 double | | 5 double | | & 2 single | | 3 double | | | | | |
| | ligation | | ligation | | ligation | | ligation | | p- | Mortality | | p- |
| | (n = 2) | | (n = 22) | | (n = 32) | | (n = 5) | | value | by Severity | | value |
| BMS ≤4 at | 2 | (100%) | 13 | (59.1%) | 3 | (9.4%) | 0 | (0.0%) | <0.001 | 15/18 | (83.3%) | <0.001 |
| day 1 (Severe | | | | | | | | | | | | |
| paralysis) | | | | | | | | | | | | |
| BMS >4 & <7.5 | 0 | (0.0%) | 6 | (27.3%) | 12 | (37.5%) | 0 | (0.0%) | | 6/18 | (33.3%) | |
| at day 1 | | | | | | | | | | | | |
| (Moderate | | | | | | | | | | | | |
| paralysis) | | | | | | | | | | | | |
| BMS ≥7.5 at | 0 | (0.0%) | 3 | (13.6%) | 17 | (53.1%) | 5 | (100%) | | 6/25 | (24.0%) | |
| day 1 (mild | | | | | | | | | | | | |
| paralysis) | | | | | | | | | | | | |
| Mortality by | 2 | (100%) | 16 | (72.72%) | 9 | (28.1%) | 0 | (0%) | <0.001 | | | |
| Ligation | | | | | | | | | | | | |

TABLE 2
Post-operative pain control agents,
concentration, dose, and frequency.

| Drug | Concentration | | Amount/Rout | Timing |
|---|---|---|---|---|
| Bupivacaine | 0.25% | | 0.1-0.2 ml | Locally at site of |
| | | | subcutaneously | incision before surgery |
| Bupre- | 0.1 | mg/kg | 1 ml | At time of surgery then |
| norphine | | | subcutaneously | as needed |
| Meloxicam | 2 | mg/kg | 1 ml | At time of surgery |
| | | | subcutaneously | then once daily for 72 |
| | | | | hours then as needed |

TABLE 3
Survival probability by study group and time.

| | | |
|---|---|---|
| Timepoint | Ligation | Sham |

| | | | |
|---|---|---|---|
| 6 | hours | 100% | 100% |
| 12 | hours | 100% | 100% |
| 2 | days | 81.2% (95% CI 62.9, 91.1) | 100% |
| 8 | days | 73.5% (95% CI 53.7, 85.9) | 100% |
| 14 | days | 63.0% (95% CI 35.6, 81.4) | 100% |

TABLE 4
Mean blood flow and velocity in the intercostal arteries and spinal
cord pre- and post-ligation and the percent change in the intercostal
and spinal cord blood flow pre-and post-ligation. All statistics are
calculated by paired t-test. Calculated using GraphPad Prism 8.4.3.

| | | Mean | SD | SEM | % CI (±) | P value |
|---|---|---|---|---|---|---|
| ICA Blood Flow | Pre-Ligation | 67.66 | 8.50 | 4.25 | 8.34 | <0.001 |
| (ml/min/100 g tissue) | Post-Ligation | 24.32 | 6.02 | 3.01 | 5.90 | |
| ICA Blood Flow % | — | −63.81 | 8.64 | 4.32 | 8.47 | — |
| Change | | | | | | |
| ICA Blood Velocity | Pre-Ligation | 4.57 | 2.09 | 1.05 | 2.05 | 0.030 |
| (m/s/100 g tissue) | Post-Ligation | 0.80 | 0.31 | 0.16 | 0.31 | |
| ICA Blood Velocity % | — | −78.14 | 13.28 | 6.64 | 13.01 | — |
| Change | | | | | | |
| Spinal Cord Blood Flow | Pre-Ligation | 31.83 | 4.79 | 2.40 | 4.70 | <0.001 |
| (ml/min/100 g tissue) | Post-Ligation | 10.40 | 4.96 | 2.48 | 4.86 | |
| Spinal Cord Blood Flow | — | −68.55 | 11.91 | 5.96 | 11.68 | — |
| % Change | | | | | | |
| Spinal Cord Blood | Pre-Ligation | 1.30 | 0.11 | 0.06 | 0.11 | <0.001 |
| Velocity (m/s/100 g | Post-Ligation | 0.49 | 0.22 | 0.12 | 0.23 | |
| tissue) | | | | | | |
| Spinal Cord Blood | — | −62.43 | 16.10 | 8.05 | 15.77 | — |
| Velocity % Change | | | | | | |

What is claimed is:

1. A method of reducing blood flow to a segment of spinal cord in a non-human vertebrate animal, the method comprising:
a) dissecting a first intercostal space to expose a first group of intercostal arteries;
b) keeping a second intercostal space intact;
c) dissecting a third intercostal space to expose a second group of intercostal arteries;
d) ligating the first group of intercostal arteries; and
e) ligating the second group of intercostal arteries, thereby reducing blood flow to a segment of spinal cord,
wherein the method comprises ligating five intercostal arteries.

2. The method of claim 1, wherein the first intercostal space and the third intercostal space are not adjacent to one another.

3. The method of claim 1, wherein the second intercostal space lies between the first intercostal space and the third intercostal space.

4. The method of claim 1, wherein the first group of intercostal arteries comprises an 8^{th}, a 9^{th}, and a 10^{th} intercostal artery.

5. The method of claim 1, wherein the second group of intercostal arteries comprises a 6^{th} and a 7^{th} intercostal artery.

6. The method of claim 1, wherein the 8^{th}, 9^{th}, and 10^{th} intercostal arteries are each ligated twice.

7. The method of claim 1, wherein the 6^{th} and 7^{th} intercostal arteries are each ligated once.

8. The method of claim 1, wherein ligating any combination of the first and second group of intercostal arteries induces ischemic spinal cord injury in the vertebrate animal, wherein a more inferior intercostal artery is ligated first, and a more superior intercostal artery is ligated last.

9. The method of claim 1, wherein ligating any combination of the first and second group of intercostal arteries mimics the effects of an ischemic brain injury in the vertebrate animal, wherein a more inferior intercostal artery is ligated first, and a more superior intercostal artery is ligated last.

10. The method of claim 1, wherein the first group of intercostal arteries and the second group of intercostal arteries branch from a descending aortic artery.

11. A method of screening for a therapeutic agent for treating a central nervous system (CNS) injury, the method comprising:
a. reducing blood flow to a segment of spinal cord in a non-human vertebrate to induce the CNS injury, wherein a first group intercostal arteries of the non-human vertebrate animal are ligated twice and wherein a second group of intercostal arteries are ligated once,
b. administering at least one therapeutic agent to the non-human vertebrate, and
c. selecting one or more therapeutic agents that increases CNS functions relative to an untreated control with the CNS injury.

12. The method of any one of claim 11, wherein the central nervous system injury is an ischemic brain injury or an ischemic spinal cord injury.

13. The method of claim 11, wherein the first group of intercostal arteries comprises an 8^{th}, a 9^{th}, and a 10^{th} intercostal artery.

14. The method of claim 11, wherein the second group of intercostal arteries comprises a 6^{th} and a 7^{th} intercostal artery.

15. The method of claim 11, wherein the method comprises ligating five intercostal arteries.

16. The method of claim 11, wherein the segment of spinal cord is a thoracic segment.

17. The method of claim 11, wherein the therapeutic agent comprises an analgesic, an anticonvulsant, an antivertigo, an anxiolytics, a sedative, an antibiotic, a CNS stimulant, a hypnotic, a muscle relaxant, or combinations thereof.

18. An engineered non-human vertebrate animal comprising reduced blood flow to a thoracic spinal cord segment, wherein a first group intercostal arteries are ligated twice and wherein a second group of intercostal arteries are ligated once, wherein the non-human vertebrate is a model for ischemic spinal cord injury or ischemic brain injury, and wherein the non-human vertebrate animal comprises a hindlimb paralysis.

19. The engineered non-human vertebrate animal of claim 18, wherein the first group of intercostal arteries comprises an 8^{th}, a 9^{th}, and a 10^{th} intercostal artery.

20. The engineered non-human vertebrate animal of claim 18, wherein the second group of intercostal arteries comprises a 6^{th} and 7^{th} intercostal artery.
